(19) United States
(12) Patent Application Publication
(10) Pub. No.: US 2015/0024376 A1
(43) Pub. Date: Jan. 22, 2015

(54) TRAP AND FLOW SYSTEM AND PROCESS FOR CAPTURE OF TARGET ANALYTES

(71) Applicant: Richard M. Ozanich, Richland, WA (US)

(72) Inventor: Richard M. Ozanich, Richland, WA (US)

(73) Assignee: BATTELLE MEMORIAL INSTITUTE, Richland, WA (US)

(21) Appl. No.: 13/946,863

(22) Filed: Jul. 19, 2013

(51) Int. Cl. G01N 33/543; G01N 33/569

(52) U.S. Cl. CPC G01N 33/54333; G01N 33/56916
USPC 435/5; 436/501; 435/7.37; 435/7.4; 435/7.35; 435/7.32; 435/6.12; 435/7.92; 435/309.1; 422/527

(57) ABSTRACT

A magnetizable trap and flow system and process are detailed that uniformly disperse paramagnetic or superparamagnetic analyte capture beads within a scaffold of magnetizable beads or other magnetizable materials in a capture zone that provides selective capture of target analytes. A magnet placed or energized in proximity to the trap may magnetize the magnetizable scaffold and secure the paramagnetic or superparamagnetic analyte capture beads in their uniformly dispersed state within the magnetizable scaffold to provide selective capture of target analytes.

### FIELD OF THE INVENTION

The present invention relates generally to bead-containing systems and methods for capturing target analytes. More particularly, the invention relates to a magnetic bead trap and flow system and process for selective capture of target analytes in a flow channel.

### BACKGROUND OF THE INVENTION

In conventional flow systems, functionalized magnetic particles typically made from iron oxide may be physically mixed with a sample to capture a target analyte of interest on the surface of the magnetic particles. Functional ligands on the surface of the magnetic particles including antibodies, oligonucleotides, lectins, proteins, or other ligands may be used to bind the target analytes of interest. To control costs, most magnetic particle-based assays use small (milligram to sub-milligram) quantities of functionalized magnetic particle materials, which limit sample sizes to a few milliliters or less on average. Conventional methods of analyte capture typically rely on passive diffusion of target analytes through the sample medium to the surface of the functionalized particles, e.g., in a microfuge tube, test tube, or microwell plate. However, the low concentration of target analytes combined with the reliance on passive diffusion of target analytes to the surface of the capture particles is inefficient, is slow (often requiring hours), and further provides less than optimal capture efficiencies (e.g., <90%) of target analytes on the surface of the particles. For example, one problem well-known to those of ordinary skill in the flow channel arts is that magnetic particles tend to clump at undesirable locations in a flow channel such as on the inner side wall of a flow channel when magnetically captured. Clumping can block the flow of analytes to the functionalized surfaces of magnetic particles, which can result in poor capture of analytes. In addition, clumped particles decrease diffusion of analytes through the particles, or diffusion may be blocked by other sample particles flowing in the clump of particles. And, clumped particles can result in only a small portion of the cross-sectional area of the flow channel being contacted by the sample. And, even when materials such as steel wool are employed to aid distribution of captured particles in a flow channel, significant clumping of particles can still occur at the initial contact point where particles and steel wool meet and/or within the steel wool due to its non-uniform structure and nominal pore size. Ultimately, some or a majority of the sample may never come in contact with the particles intended to capture the target analytes of interest. Accordingly, new approaches are needed that enhance analyte capture in magnetic particle systems. The present invention addresses these needs.

### SUMMARY OF THE PRESENT INVENTION

The present invention includes a trap and flow system and process that provide selective capture of target analytes. The system may include a magnetizable scaffold comprised of magnetizable scaffold beads in a capture zone or trap. The system may further include paramagnetic or superparamagnetic analyte capture beads of a selected size smaller than the magnetizable scaffold beads that are distributed within the magnetizable scaffold. Surfaces of the analyte capture beads may be functionalized to capture selected analytes when a sample containing the analytes is introduced through the scaffold in the capture zone.

One or more magnets may be positioned to magnetize the magnetizable scaffold and secure the analyte capture beads in the capture zone. The magnets may be positioned on a translational or reciprocating stage. The translational stage may be configured to position the magnet in a first position proximate the capture zone that magnetizes the scaffold and secures the paramagnetic or superparamagnetic analyte capture beads in a dispersed state under a fluid pressure in the scaffold in the capture zone. The translational stage may also include a second position a selected distance removed from the capture zone that releases analyte capture beads from the scaffold in the capture zone for collection of the captured analytes.

The present invention also includes a method for selective capture of target analytes. The method may include distributing a quantity of magnetizable scaffold beads in the capture zone or trap to form a scaffold.

In some applications, magnetizable scaffold beads may include or be composed of such materials as solid glass, solid semi-synthetic organic polymers, synthetic organic polymers including, but not limited to, e.g., polystyrene, polyethylene, nylon, fluoro-containing polymers such as polytetrafluoroethylene (PTFE) also known as TEFLON® (DuPont, Wilmington, Del., USA), and combinations of these various materials.

In some applications, magnetizable scaffold beads may have a magnetizable center or core and may include, be composed of, or be coated with in whole or in part such materials as solid glass, silica of various pore sizes, semi-synthetic organic polymers, synthetic organic polymers such as, e.g., polystyrene, polyethylene, nylon, fluoro-containing polymers such as polytetrafluoroethylene (PTFE) also known as TEFLON® (DuPont, Wilmington, Del., USA), and combinations of these various materials.

In some applications, magnetizable scaffold beads may include or may be composed of a metal such as nickel, cobalt, iron, or a combination of these metals. In some applications, magnetizable scaffold beads may be solid metal beads. In some applications, magnetizable scaffold beads may be metal-coated beads coated with a magnetizable metal. In some applications, magnetizable scaffold beads may be metal-coated spheres composed of hollow glass or hollow polystyrene coated with a magnetizable metal. In some applications, magnetizable scaffold beads may be composed of hollow metal spheres.

Magnetizable scaffold beads may include various sizes in the range from about 1 nm to about 10,000 nm. In some applications, magnetizable scaffold beads may include a size in the range from about 5 μm to about 150 μm. In some applications, magnetizable scaffold beads may include a size in the range from about 150 μm to about 10 mm.

The method may also include dispersing a quantity of analyte capture beads into the capture zone of a size equal to or smaller than the magnetizable scaffold beads so that the analyte capture beads may distribute uniformly through the stack of magnetizable scaffold beads. Analyte capture beads may be paramagnetic or superparamagnetic beads that include or are composed of iron oxide dispersed in a polymer matrix. Analyte capture beads may also be paramagnetic or superparamagnetic beads that include or are composed of iron oxide with a shell comprised of inert materials including, e.g., graphite, grapheme, polymers, silica, or other inert materials that improve compatibility with various sample matrices or target analytes, or otherwise improve particle dispersion or target analyte capture.

The method may also include magnetizing the magnetizable scaffold beads in the capture zone to trap the analyte capture beads in their dispersed state in the scaffold in the capture zone. The method may also include flowing a sample through the capture zone to trap one or more target analytes when present in the sample on the surface of the paramagnetic or superparamagnetic analyte capture beads dispersed within the scaffold.

Other magnetizable materials including, e.g., metal foams, wools, meshes, wires, and combinations of these various materials may be introduced into the capture zone along with the magnetizable scaffold beads that form the trap to maintain pores of a selected size or to maintain a selected porosity in the scaffold that assist in dispersing analyte capture beads through the scaffold in the capture zone or allow passage of selected materials. Magnetizable materials may include a porosity of from about 100 nm to about 10 mm.

Magnetizable scaffold beads may be restrained in the capture zone with a rotatable rod positioned adjacent to the capture zone in the flow channel. In some applications, the rotatable rod may be positioned, e.g., at an exit end of the capture zone. The rod may include a beveled (angled) face that when placed in the flow channel forms the bottom of the capture zone. In a 1^{st} position, the beveled face of the rotatable rod may restrain (trap) the magnetizable scaffold beads in the capture zone. In a 2^{nd} position, the beveled face of the rotatable rod can release the magnetizable scaffold beads from the capture zone.

In some applications, paramagnetic or superparamagnetic analyte capture beads may be dispersed into a volume of a carrier fluid approximately equal to the volume of the capture zone and introduced into the capture zone in the dispersed state. In some applications, paramagnetic or superparamagnetic analyte capture beads may be dispersed and introduced in a volume of a carrier fluid that is less than or equal to the volume of the magnetizable scaffold beads within the capture zone.

Paramagnetic or superparamagnetic analyte capture beads may have a selected surface chemistry or functionalization configured to capture a selected target analyte or target analytes thereon. Surfaces of the paramagnetic or superparamagnetic analyte beads may be functionalized with one or more components selective for target analytes including, but not limited to, e.g., antibodies, oligonucleotides, DNA, RNA, aptamers, haploids, lectins, carbohydrates, proteins, chelating agents, silica, hydroxyapatite, and combinations of these various components. In some applications, surfaces of analyte capture beads may be functionalized to capture a single analyte. In some applications, surfaces of paramagnetic or superparamagnetic analyte capture beads may be functionalized to capture a single analyte may be introduced into the capture zone. In some applications, two or more different types of paramagnetic or superparamagnetic analyte capture beads each functionalized to capture a different analyte may be introduced into the capture zone to capture different analytes.

In some applications, the surface may be functionalized to capture two or more analytes. In some applications, paramagnetic or superparamagnetic analyte capture beads functionalized to capture two or more analytes may be introduced into the capture zone.

Paramagnetic or superparamagnetic analyte capture beads may include a size that is about 100 to 1000 times smaller than the magnetizable scaffold beads. Paramagnetic or superparamagnetic analyte capture beads may be dispersed into the capture zone as a suspension in a carrier fluid. Paramagnetic or superparamagnetic analyte capture beads may be dispersed into the capture zone prior to flowing the sample therein. Paramagnetic or superparamagnetic analyte capture beads may also be dispersed into the capture zone simultaneously with the sample containing target analytes.

Magnetizing the magnetizable scaffold beads in the capture zone may be performed with one or more magnets of selected shapes or selected types. Magnets may be permanent magnets, a Halbach array of permanent magnets, electromagnets, ring magnets, tapered magnets, fixed magnets, multiple magnets with non-magnetic spacers, and combinations of these various types of magnets.

In some applications, magnetizing the magnetizable scaffold beads may be performed with one or more permanent magnets. In some applications, magnetizing the magnetizable scaffold beads may be performed with a permanent magnet such as a neodymium magnet that includes a rare earth material such as NdFeB. In some applications, magnetizing the magnetizable scaffold beads may be performed with an electromagnet. In some applications, magnetizing the magnetizable scaffold beads may be performed with a Halbach Array. In some applications, magnetizing the magnetizable scaffold beads may be performed with a ring magnet.

Flowing the sample through the capture zone may include flowing the sample at a flow rate selected to provide a residence time sufficient for capture of the target analyte or target analytes on the surface of the paramagnetic or superparamagnetic analyte capture beads that are dispersed in the capture zone.

Magnetizing the magnetizable scaffold beads may include applying a magnetic field with a magnet in a direction orthogonal to the flow of the carrier fluid or sample within the capture zone of the flow channel.

In some applications, the magnet may be a cylindrical (ring) magnet, a rectangular magnet, or a square magnet that delivers a magnetic field in a direction orthogonal to the flow of the carrier fluid in the capture zone.

In some applications, the magnet may be a ring magnet or a solenoid-based electromagnet that delivers a magnetic field in a direction parallel to the flow of the carrier fluid in the capture zone.

In some applications, one or more magnets may be stacked with non-magnetic spacers between the magnets to increase the strength and uniformity of the magnetic field introduced into the capture zone or flow channel.

Magnets may also be placed on a displaceable or reciprocating translation stage to position the magnet proximate the capture zone that magnetizes both the magnetizable scaffold beads and the paramagnetic or superparamagnetic analyte capture beads for capture of selected target analytes.

In some applications, magnetizing the magnetizable scaffold beads may be performed with a magnet or magnets in a magnet assembly. The magnet assembly may include a 1^{st} position that positions the magnet(s) to allow flow of magnetizable scaffold beads through the flow channel into the capture zone and a 2^{nd} position that positions the magnet(s) so as to retain magnetizable scaffold beads in the capture zone under a fluid pressure provided by the carrier fluid.

Paramagnetic or superparamagnetic analyte capture beads may include various functionalized surfaces known in the art that are selective for target analytes of interest. Target analytes may include, but are not limited to, e.g., proteins, lipids, antigens, viruses, bacteria, spores, oocytes, mammalian cells, sperm cells, radionuclides, heavy metals, and combinations of these various analytes. The analyte may also be a biothreat agent such as anthrax (Bacillus anthracis), botulism (Clostridium botulinum toxin), plague (Yersinia pestis), smallpox (variola major), tularemia (Francisella tularensis), viral hemorrhagic fevers (filoviruses [e.g., Ebola, Marburg] and arenaviruses [e.g., Lassa, Machupo]), brucellosis (Brucella species), epsilon toxin of Clostridium perfringens, food safety threats (e.g., Salmonella species, Listeria species, Listeria monocytogenes, E-coli species, Escherichia coli (E-coli) O157:H7, Shigella, glanders (Burkholderia mallei), Melioidosis (Burkholderia pseudomallei), Psittacosis (Chlamydia psittaci), Q fever (Coxiella burnetii), ricin toxin from Ricinus communis (castor beans), Staphylococcal enterotoxin B, Typhus fever (Rickettsia prowazekii), viral encephalitis (alphaviruses [e.g., Venezuelan equine encephalitis, eastern equine encephalitis, western equine encephalitis]), water safety threats (e.g., Vibrio cholerae, Cryptosporidium parvum), or combinations of any of these biothreats.

Samples may include a volume or size between about 1 microliter (μL) and about 10,000 liters (L). Sample volumes may also be between about 10 μL and about 10 milliliters (mL). Sample volumes may also be between about 10 milliliters (mL) and about 1000 milliliters (mL). No limitations are intended.

The method may further include collecting paramagnetic or superparamagnetic analyte capture beads with the captured analyte thereon from the flow channel for analysis of the target analyte.

The purpose of the foregoing abstract is to enable the United States Patent and Trademark Office and the public generally, especially the scientists, engineers, and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

### BRIEF DESCRIPTION OF THE DRAWINGS

FIGS. 1a-1b show two embodiments of a sequential injection system for selective capture of target analytes.

FIGS. 2a-2b illustrate a rotatable rod in closed and open position, respectively, for retaining and manipulating magnetizable (scaffold) beads in concert with the present invention.

FIGS. 3a-3d show exemplary types of magnetizable scaffold beads.

FIG. 4 presents an exemplary process for selective capture of target analytes in accordance with the present invention.

FIGS. 5a-5e illustrate embodiments of the process of the present invention.

### DETAILED DESCRIPTION

A magnetizable trap and flow system and process are detailed for selective capture of target analytes. Other supporting aspects of Biodetection-Enabling Analyte Delivery Systems (BEADS) are detailed in U.S. Pat. Nos. 6,136,197, 6,159,378, 6,645,377, 6,780,326, 7,001,522, and 7,090,774, assigned to Battelle Memorial Institute, which references are incorporated herein in their entirety. The following description details a best mode of the present invention. While the invention is susceptible of various modifications and alternative constructions, it will be clear from this description that the invention is not limited to these illustrated embodiments but that the invention also includes a variety of modifications and embodiments thereto. The invention is to cover all modifications, alternative constructions, and equivalents falling within the spirit and scope of the invention as defined in the claims. Therefore the present description should be seen as illustrative and not limiting.

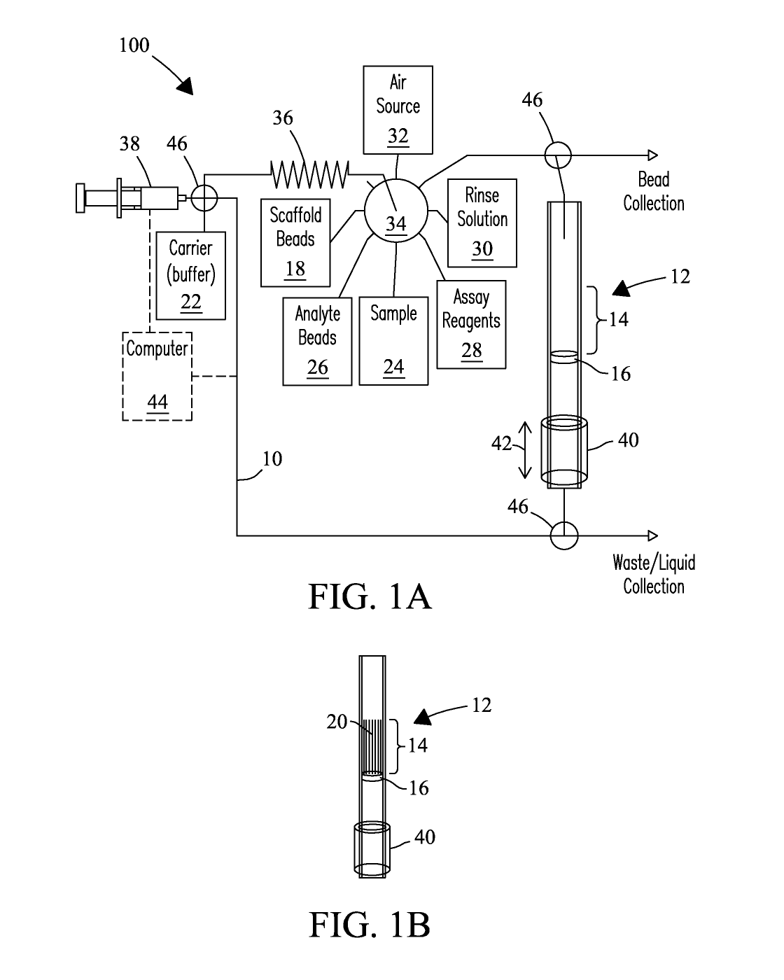

FIG. 1a shows a magnetizable trap and flow system 100 for selective capture of target analytes according to a preferred embodiment of the present invention. The magnetizable trap may be constructed with magnetizable scaffold beads configured to provide capture of target analytes. The term “magnetizable” as used herein refers to materials that exhibit a magnetic field less than one Gauss, or, in the absence of an externally applied magnetic field, are not attracted to or will not adhere to another structure, entity, or body via magnetic forces. In the presence of an applied magnetic field, such materials are attracted to, attach to, or adhere to another structure, entity, or body. System 100 may include a flow channel 10 constructed of non-magnetic tubing of various selected and non-limiting diameters. System 100 may include a flow trap (trap) 12 introduced at a selected location in flow channel 10. Trap 12 may include a capture zone 14 configured for capture of target analytes as described further herein. In some embodiments, trap 12 may include an inner diameter larger [e.g., ½-inch (1.3 cm)] than the diameter [e.g., ⅛-inch (0.3 cm)] of flow channel 10, but diameters are not limited thereto. Diameters may be scaled to meet demands of the intended applications. Thus, no limitations are intended.

Magnetizable scaffold beads 18 may be introduced into capture zone 14 to form a magnetizable scaffold 18 in trap 12. Paramagnetic or superparamagnetic analyte capture beads 26 may be introduced in capture zone 14 after introduction of magnetizable scaffold beads 18. “Paramagnetic” and “superparamagnetic” as these terms are used herein refer to materials that may become magnetized in the presence of an externally applied magnetic field. Paramagnetic and superparamagnetic materials may have a small positive susceptibility when a magnetic field is applied. The magnetic moment induced by the applied field may be linear in field strength. Such materials do not retain their magnetic properties when the magnetic field is removed. Paramagnetic or superparamagnetic analyte capture beads 26 may be of a size that permits them to disperse uniformly within magnetizable scaffold 18 into interstitial spaces (not shown) located between adjacent scaffold beads 18 in capture zone 14.

Other magnetizable scaffolding materials 20 including, but not limited to, e.g., metal foams, wools, meshes, wires, and combinations of these various materials may also be introduced into capture zone 14 in trap 12 alone or along with magnetizable scaffold beads 18 to provide selected porosities in scaffold 18. And, when used, magnetizable scaffolding materials 20 may assist in the uniform dispersal of paramagnetic and superparamagnetic analyte capture beads within the magnetizable scaffold 18.

In various embodiments, surfaces of paramagnetic or superparamagnetic analyte capture beads 26 may be functionalized as described further herein to provide selective capture of target analytes when a sample 24 containing the target analytes is introduced through the scaffold in the capture zone (trap) 14.

In various embodiments, a retention filter 16 with a selected porosity may be positioned at the exit end of trap 12 to retain magnetizable scaffold beads 18 within trap 12 and allow carrier fluids 22 and samples 24 containing potential target analytes (not shown), and, in some cases analyte capture beads 26 and other fluids (rinse fluids, detergent fluids, disinfectant fluids, etc.) to flow through trap 12. Filters suitable for use include, but are not limited to, e.g., mesh filters, polymer filters, metal foam filters, and like filtering materials.

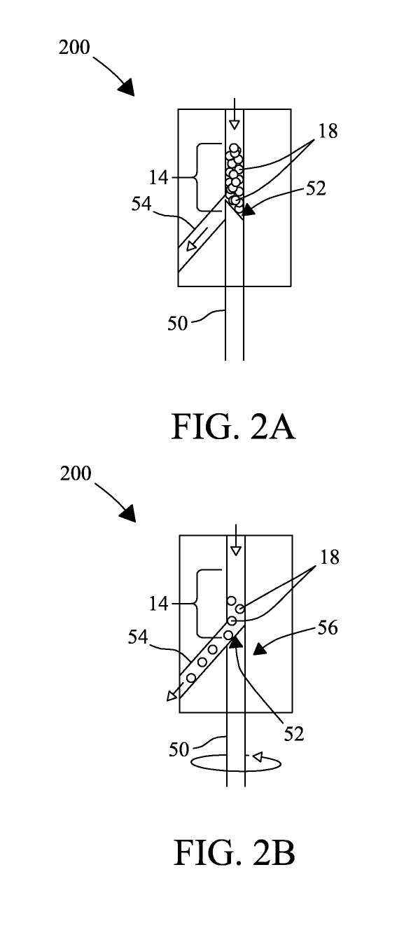

In some embodiments, a rotatable rod (described further herein in reference to FIG. 2) may be positioned at the exit end of trap 12 to retain magnetizable scaffold beads 18 introduced as a primary scaffold 18 within trap 12 that allows carrier fluids 22 and samples 24 containing potential target analytes, and other fluids to flow through trap 12. Trap 12 may be operated at ambient temperature, or at lower and higher temperature conditions. No limitations are intended.

Carrier fluids (or buffers) 22, samples 24, various assay reagents 28 (e.g., detergents, bleach, disinfectants) as well as rinse solutions 30, and gases 32 (e.g., air), including suspensions and mixtures of these various components may be aspirated, e.g., through a multi-position selection valve 34 into a holding coil 36 and dispensed back through multi-position selection valve 34 into bead trap 12. Types and number of reagent and fluid sources coupled through multi-position selection valve 34 are not limited. For example, in various embodiments, analyte beads 26 with various functionalized surfaces may be coupled to selection valve 34 for individual or simultaneous introduction into bead trap 12. In other embodiments, magnetizable beads 18 of various sizes may be coupled to selection valve 34 for individual or simultaneous introduction into bead trap 12. In some embodiments, magnetizable scaffolding materials 20 may be preinstalled (i.e., not introduced via multi-position valve 34) into capture zone 14 within trap 12. In some embodiments, magnetizable scaffolding materials 20 may be introduced via multi-position valve 34 into capture zone 14 within trap 12. All materials, reagents, fluids, analyte capture beads, and magnetizable beads, including different ones or multiples of same, as will be configured and employed by those of ordinary skill in the art in view of the disclosure are within the scope of the present invention. No limitations are intended.

A pump 38 such as a syringe pump, peristaltic pump, or diaphragm pump, or other devices that propel fluids with, e.g., pressure or vacuum may be periodically or partially filled with a carrier solution 22 or rinse solution 30 to maintain a clean side to flow system 100. In the figure, a holding coil 36 can prevent liquids other than the desired carrier solution 22 or rinse solution 30 from contacting pump 38. In addition, one or more disinfection and/or cleaning solutions (not shown) may be aspirated through multi-position selection valve 34.

System 100 may further include a magnet 40 or magnet assembly 40 that positions, or may be positioned proximate or adjacent to capture zone 14 to provide a magnetic field (not shown) across or within capture zone 14. When positioned proximate or adjacent to trap 12 in capture zone 14, magnet 40 may magnetize scaffold beads (scaffold) 18 and/or other magnetizable scaffold materials 20 present within trap 12. The magnetization may also secure analyte beads 26 in their distributed (i.e., uniformly dispersed) state in the magnetizable scaffold beads (scaffold) 18 and/or other magnetizable scaffold materials 20 in trap 12. When magnet 40 is not positioned proximate or adjacent to trap 12, scaffold beads 18 and/or other magnetizable scaffold materials 20 in capture zone 14 do not retain their magnetization, which may release or allow analyte capture beads 26 to flow from trap 12. In some embodiments, with the magnetic field off, analyte capture beads 26 can be separated from scaffold beads 18 by flowing the analyte capture beads 26 through the retaining filter 16 when pores of the scaffold 18 and filter 16 are sufficiently large. In some embodiments, with the magnetic field in place, analyte capture beads 26 can be separated from the scaffold beads 18 by flowing carrier fluid in an upward direction through trap 12 at a flow rate sufficient to remove the analyte capture beads 26, but not dislodge the scaffold beads 18.

Paramagnetic or superparamagnetic analyte capture beads 26 can be delivered into trap 12 in capture zone 14 as a uniform suspension and locked in a dispersed or uniformly distributed state by applying a magnetic field across capture zone 14. When dispersed within the scaffold 18 of magnetizable beads in capture zone 14, paramagnetic or superparamagnetic analyte capture beads 26 facilitate and enhance mass transport of analytes through the scaffold 18. Samples 24 introduced into capture zone 14 may then be actively flowed past analyte capture beads 26 within scaffold 18 dispersed in trap 12, which provides efficient capture of target analytes from samples 24 by a factor of at least two-fold to four-fold when compared to conventional systems known in the art. System 100 is well suited for capture of target analytes present within sample volumes that are both small and large. In some embodiments, sample volumes are between about 10 milliliters to about 100 milliliters or more. Smaller and larger sample volumes may also be analyzed as detailed herein. No limitations are intended.

Target analytes captured from samples 24 may be retained on the functionalized surface of paramagnetic or superparamagnetic analyte capture beads 26 until the analyte capture beads 26 are released and/or collected from capture zone (trap) 14. Shapes of trap 12 and flow cell 10 are not limited. For example, capture zone 14 may be as a column or other cylindrically-shaped format, or in other formats that reduce clumping and better facilitate dispersion of paramagnetic or superparamagnetic analyte capture beads 26 through the magnetizable scaffold 18 present in capture zone 14.

In some embodiments, a ring magnet 40 may be employed as the magnetic field source. Ring magnet 40 may be mounted, e.g., on a translation stage 42 or other displacement device or means (not shown) that allows magnet 40 to be positioned (e.g., under manual, electronic, pneumatic, or computer control) to lock or removed to release analyte beads 26 from trap 12 in capture zone 14. For example, when positioned in place, ring magnet 40 may be centered such that it surrounds capture zone 14, locking analyte beads 26 within trap 12. When ring magnet 40 is displaced or positioned away from capture zone 14, the magnetic field delivered across capture zone 14 is removed, which serves to release analyte beads 26 from within the scaffolding of magnetizable beads 18, permitting analyte beads 26 to be collected for analysis of their captured target analytes.

In some embodiments, an electromagnet 40 detailed, e.g., by Holman et al. in U.S. Pat. No. 6,159,378 may be positioned proximate or adjacent to capture zone 14 as a magnetic field source, which reference is incorporated herein in its entirety. When energized, electromagnet 40 may then deliver a suitable electric field across capture zone 14 allowing capture of analyte beads 26 within trap 12 and thus capture of target analytes. When de-energized, analyte beads 26 may be released from trap 12, allowing collection of analyte beads 26 for analysis of captured target analytes.

In yet another embodiment, a fixed magnet 40 detailed, e.g., by Holman et al. in U.S. Pat. No. 6,159,378 with flow controls may be employed, which reference is incorporated herein in its entirety.

Trapped paramagnetic or superparamagnetic analyte capture beads 26 may be subsequently collected and submitted to various detection assays (e.g., mouse or other bioassays) for detection, determination, culturing, agar plating, and/or analysis of captured target analytes.

System 100 may further be automated with a computer or computers 44 that provide automation of devices including, but not limited to, e.g., pumps 38, multi-position selection valves 34, or, e.g., provide automation of the selection of one or more of: scaffold beads 18, carrier solution 22, various samples 24, one or more sizes or types of analyte capture beads 26 (e.g., with different functionalized surfaces for capture of different target analytes), assay reagents 28, rinse solutions 30, and etc., including controlling such parameters as fluid flow rates, fluid volumes, fluid flow directions (e.g., to re-suspend settled analyte capture beads 26 and/or magnetizable scaffold beads 18 in the source vessel), delivery times of selected and various reagents, contact times for selected reagents within the trapping region, and controlling timing of the opening and closing of trap 12, or other components within system 100 as will be understood and appreciated by those of ordinary skill in the analytical systems arts. No limitations are intended.

Flow channel 10 and trap 12 may be constructed of any non-magnetic materials employed for transport of liquids, fluids, reagents, samples, carrier fluids, rinsing agents, and beads including, but not limited to, e.g., capillaries, pipes, conduits, and tubing. Materials suitable for construction of capillaries, pipes, conduits, and tubing include, but are not limited to, e.g., polymers, plastics, resins, non-magnetic metals, glass, ceramics, and combinations of these various materials. Preferred materials are easily cleaned, easily replaced following use, and cost-effective, but materials are not intended to be limited.

FIG. 1b shows an alternate configuration for magnetizable trap 12 (described previously in reference to FIG. 1a) in capture zone 14. In this configuration, magnetizable trap 12 may be constructed or configured with other magnetizable materials 20 other than magnetizable scaffold beads (FIG. 1a) and positioned above filter 16. Other magnetizable materials 20 may include, but are not limited to, e.g., metal foams, metal wools, metal meshes (e.g., nickel metal meshes), metal beads, metal particles, and metal wires. Other magnetizable materials 20 described herein may be pre-formed, pre-packed, or pre-introduced in magnetizable trap 12 to provide capture of target analytes. Ring magnet 40 is shown positioned adjacent capture zone 14 prior to introducing paramagnetic or superparamagnetic analyte capture beads (FIG. 1a).

### Rotatable Rod System

FIGS. 2a-2b illustrate a rotatable rod system 200 adapted for physical restraint, retention, and release of magnetizable scaffold beads 18 and, optionally, paramagnetic or superparamagnetic analyte capture beads 26 introduced to trap 12 within flow channel 10. Rod system 200 employed herein for retention and manipulation of scaffold beads 18 may be adapted from a system for retention and manipulation of non-magnetic particles described, e.g., by Egorov et al. in U.S. Pat. Nos. 6,136,197, 6,645,377, and 6,780,326, which references are incorporated in their entirety herein.

System 200 may include a rod 50 positioned at the exit end of trap 12 within capture zone 14 within flow channel 10. Rod 50 may include a beveled face 52 that when rotated axially within channel 10 may provide a closed trapping condition or an open flow condition. FIG. 2a shows rod 50 with a beveled face 52 oriented away from fluid (exit) channel 54 in a bead trapping (i.e., closed) position. In this position, beveled face 52 retains magnetizable beads 18 when introduced into trap 12. Fluid channel 54 may be positioned adjacent the tip of beveled face 52 of beveled rod 50 to allow fluids to flow past and around the beveled face 52 of rod 50 (e.g., when closed) that allows magnetizable scaffold beads 18 to flow into trap 12 and stack when introduced into trap 12 forming the magnetizable scaffold 18. With the scaffold beads 18 in place in trap 12, surface functionalized paramagnetic or superparamagnetic analyte capture beads 26 may then be introduced into trap 12, as detailed further herein. Samples 24 containing target analytes may then be actively flowed past analyte capture beads 26 that are distributed uniformly within the scaffold beads (scaffold) 18 in trap 12 to enhance capture of target analytes on surfaces of the analyte capture beads 26. Once target analytes are captured, analyte capture beads 26 can be separated from scaffold beads (scaffold) 18 by removing the magnetic field (described previously in reference to FIG. 1) that releases the analyte capture beads 26 to flow past or around rod 50 through fluid (exit) channel 54.

In an alternate approach, with the magnetic field in place, analyte capture beads 26 can be separated from scaffold beads 18 by increasing the rate of flow of carrier fluid in a reverse flow direction at a flow rate sufficient to overcome the magnetic attraction of the capture beads 26 that displaces them but does not displace the scaffold beads 18.

In another alternate approach, scaffold beads 18 and analyte capture beads 26 can be released together. FIG. 2b shows rotatable rod 50 with beveled face 52 in an open (flowing) position. Removing the magnetic field and rotating rod 50 so that face 52 is aligned with exit channel 54 releases both scaffold beads 18 and analyte capture beads 26 from trap 12 for collection and/or separation downstream from trap 12. Bead separation may be performed after collection using physical, magnetic, or other separation processes known in the separation arts or in concert with buoyancy differences between beads as detailed further herein.

Rod system 200 is effective at retaining magnetizable scaffold beads 18, capturing analyte capture beads 26 in a distributed state, improving contact and reaction rates of various reagents through the scaffold of scaffold beads 18 and analyte capture beads 26 within trap 12, and capturing target analytes from samples 24 with a high efficiency. In operation, for example, rod system 200 provides a concentrated co-location (i.e., with magnetizable scaffold beads 18) of distributed analyte capture beads 26 that enhances mass transport of target analytes from the samples 24 to the surfaces of the analyte capture beads 26 that yields at least a 4-fold greater sensitivity and faster capture times when compared with manual mixing of samples 24 with analyte capture beads 26 for capture of target analytes.

As will be appreciated and understood by those of ordinary skill in the art, system 200 may be operated manually or be automated in conjunction with, e.g., a computer or computer control as described herein. Thus, no limitations are intended. Employing rotating rod system 200 to control introduction and release of scaffold beads 18 and analyte capture beads 26 within bead trap 12 has distinct advantages. First, rotating rod system 200 may provide more efficient clearing and removal of non-retained materials and reagents from magnetizable scaffold beads 18 positioned within bead trap 12, which can reduce backgrounds and interferences in detection signals obtained during detection and analysis of target analytes. Rod system 200 can also provide reproducible and consistent results as all samples 24 are handled similarly, which can reduce variability in sample handling and processing. And, as discussed herein, rotating rod system 200 can improve mass transport within bead trap 12 for capture of target analytes, providing a greater sensitivity and faster assays compared with conventional flow systems. And, rotating rod system 200 can also be used to perfuse large sample volumes (e.g., for pre-concentration applications) through bead trap 12. All configurations as will be implemented by those of ordinary skill in the art in view of this disclosure are within the scope of the invention. No limitations are intended.

### Magnetizable Scaffold Beads

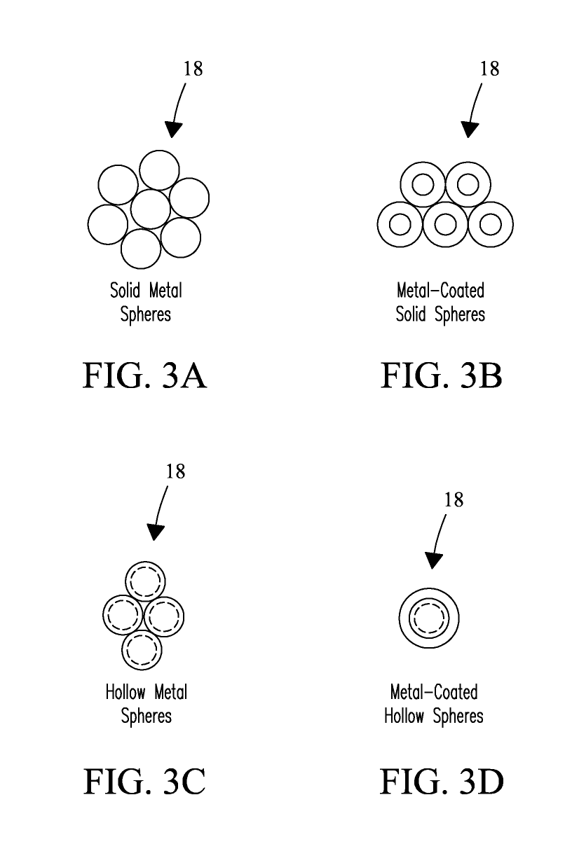

FIGS. 3a-3d show various and exemplary forms of magnetizable scaffold beads 18 suitable for use as a scaffold 18 in the bead trap (FIG. 1a) within the capture zone (FIG. 1a). Magnetizable scaffold beads 18 may take the form of, e.g., solid metal-containing spheres (FIG. 3a); metal-coated solid metal-containing spheres (FIG. 3b); hollow metal-containing spheres (FIG. 3c); metal-coated, hollow metal-containing spheres (FIG. 3d); and other suitable forms including metal-coated solid non-metal containing spheres; metal-coated, hollow non-metal containing spheres, including combinations of these various types and forms. No limitations are intended. Magnetizable scaffold beads 18 may further include, or be constructed of materials such as polymers, hydrogels, glasses, metals, ceramics, and combinations of these various materials. Magnetizable scaffold beads 18 may also be spherical or non-spherical. Magnetizable scaffold beads 18 may also take the form of, e.g., metal-coated solid non-metal containing spheres such as metal-coated solid glass spheres or metal-coated hollow glass spheres. Magnetizable scaffold beads 18 may also take the form of metal-coated non-metal containing spheres constructed, e.g., of polymers such as polystyrene that are coated with a selected metal. Magnetizable scaffold beads 18 of various forms can provide buoyancy that assists distribution in the bead trap (FIG. 1a) within the capture zone (FIG. 1a) for selected applications, and can also assist in the separation of scaffold beads 18 from paramagnetic or superparamagnetic analyte capture beads (FIG. 1a). Once separated, target analytes captured by the paramagnetic or superparamagnetic analyte capture beads may be analyzed, and scaffold beads 18 and/or analyte capture beads may be re-used. Magnetizable scaffold beads 18 used as scaffolds 18 in capture zone may also include or be composed of metals such as, e.g., nickel (Ni), cobalt (Co), magnesium (Mg), molybdenum (Mo), tantalum (Ta), lithium (Li), dysprosium (Dm), gadolinium (Gd), combinations thereof, and alloys of these various metals.

Magnetizable (scaffold) beads 18 may be of any suitable or selected size such that when introduced into the trap form a scaffold 18 (e.g., a stack of beads). The scaffold of magnetizable beads (scaffold) 18 may include interstitial spaces between the beads 18 that permits smaller analyte capture beads described hereafter to distribute uniformly through scaffold 18 within the bead trap. In some embodiments, magnetizable beads 18 may include various individual sizes and dimensions that permit the distribution of smaller analyte beads within the magnetizable bead scaffold 18. In some embodiments, magnetizable beads 18 may include various mixtures of sizes and dimensions that permit the distribution of smaller analyte beads within the magnetizable bead scaffold 18. In various embodiments, size may be between about 1 nm and about 10,000 nm. In some embodiments, size may be between about 1 nm and about 3000 nm. In some embodiments, size may be between about 5 μm and about 150 μm. In some embodiments, size may be between about 150 μm and about 10 mm. No limitations are intended.

In some embodiments, magnetizable scaffold beads 18 may be spherical (e.g., 10-30 microns) and uniform to allow free flow of carrier fluids, reagents, wash solutions, and sample solutions to flow through the interstitial spaces located between beads 18 in the capture zone. In some embodiments, packed columns containing different sizes of metal-containing magnetizable beads 18 can be used as a scaffold in the bead trap, which can allow various and different sizes of paramagnetic or superparamagnetic analyte beads to be employed for trapping and capture of target analytes in the bead trap. No limitations are intended.

As shown in FIG. 1b, other magnetizable materials 20 may be used as a scaffold 20 in trap 12 in place of magnetizable scaffold beads (FIG. 1a). Other magnetizable scaffold materials 20 may provide a suitable porosity (i.e., pore size) that allows analyte capture beads 26 to disperse and distribute uniformly within the scaffold 20. Other magnetizable materials 20 suitable for use as a scaffold 20 in trap 12 may include, but are not limited to, metal-containing pillars, metal-containing wires, metal-containing filaments, metal-containing meshes of various mesh sizes, metal-containing screens, metal-containing filters, metal-containing wools, metal-containing fibers, metal-containing foams with various porosities or pore sizes, including combinations of these various magnetizable materials. No limitations are intended.

In some embodiments, other magnetizable materials 20 may include metal-containing wools. Metal wools may include a metal including, but not limited to, e.g., nickel (Ni), cobalt (Co), magnesium (Mg), molybdenum (Mo), tantalum (Ta), iron (Fe), combinations of these metals, and alloys thereof. Metal wools when used as a magnetizable material 20 in capture zone 14 permit capture of paramagnetic or superparamagnetic analyte capture beads 26 of various sizes when magnetized.

In some embodiments, other magnetizable materials 20 may include nickel foams, nickel wire meshes, nickel beads, nickel particles, and nickel wires. In some embodiments, other magnetizable materials 20 may be composed of or include up to about 100% nickel by weight.

In some embodiments, other magnetizable materials 20 may take the form of packed or pre-packed columns, or formed or pre-formed columns, e.g., for one-time use or for rapid exchange following use. Packed or pre-packed columns may include nanoscale or microscale metal beads or particles of a spherical or non-spherical shape that include or are composed of, e.g., selected metals described herein, selected packing densities, and sizes selected to provide interstitial spaces (interstices) of various dimensions between the beads in the packed columns that disperses analyte capture beads uniformly when introduced to the scaffold 20. In some embodiments, quantity of magnetizable materials 20 that constitute scaffold 20 within trap 12 may have a volume that is equal to or greater than the volume of scaffold beads described previously in reference to FIG. 1a. In some embodiments, other magnetizable materials 20 that constitute scaffold 20 within trap 12 may have a volume that is less than or equal to the volume of scaffold beads described previously in reference to FIG. 1a. No limitations are intended.

### Paramagnetic or Superparamagnetic Analyte Capture Beads

Bead trap 12 may be configured to provide capture of one or more selected analytes by uniformly dispersing paramagnetic or superparamagnetic analyte capture beads 26 throughout the scaffold of scaffold beads 18 within capture zone 14. Analyte capture beads 26 may be uniformly suspended in a carrier fluid 22 or other fluid and introduced into capture zone 14 at any suitable concentration or volume. In some embodiments, volume of analyte capture beads 26 introduced into trap 12 may be equivalent to the volume of open (interstitial space) space between the magnetizable scaffold beads 18 and/or other magnetizable materials present in trap 12. Analyte capture beads 26 may be of any suitable size or dimension smaller than the scaffold beads 18 to allow the analyte capture beads 26 to uniformly disperse throughout the scaffold of scaffold beads 18 within trap 12.

Sizes for paramagnetic or superparamagnetic analyte capture beads 26 are not limited. Sizes are selected that allow the analyte capture beads 26 to flow into open interstitial spaces located between adjacent magnetizable scaffolding beads 18 positioned in capture zone 14 or to flow into porous magnetizable scaffolding materials 20 described previously herein. In some embodiments, sizes of the paramagnetic or superparamagnetic analyte capture beads 26 may be selected between about 25 nanometers (nm) and about 10 microns (μm).

Analyte capture beads 26 described herein may be configured with any functionalized surface known in the art for selective capture of target analytes. Analyte capture beads 26 suitable for use in concert with the present invention are available commercially, e.g., from Life Technologies Inc. (formerly Dynal) (Carlsbad, Calif., USA); Promega (Madison, Wis., USA); Polysciences, Inc. (Warrington, Pa., USA); Chemicell GmbH (Berlin, Germany); and like vendors. Surfaces of the analyte capture beads may be functionalized for selective capture of analytes with materials employed widely in biotechnology and biodetection applications including, but are not limited to, e.g., antibodies, antigens, oligonucleotides, lectins, carbohydrates, silica, hydroxyapatite, other analyte-specific ligands, including combinations of these various materials. No limitations are intended.

### Process for Selective Capture of Target Analytes

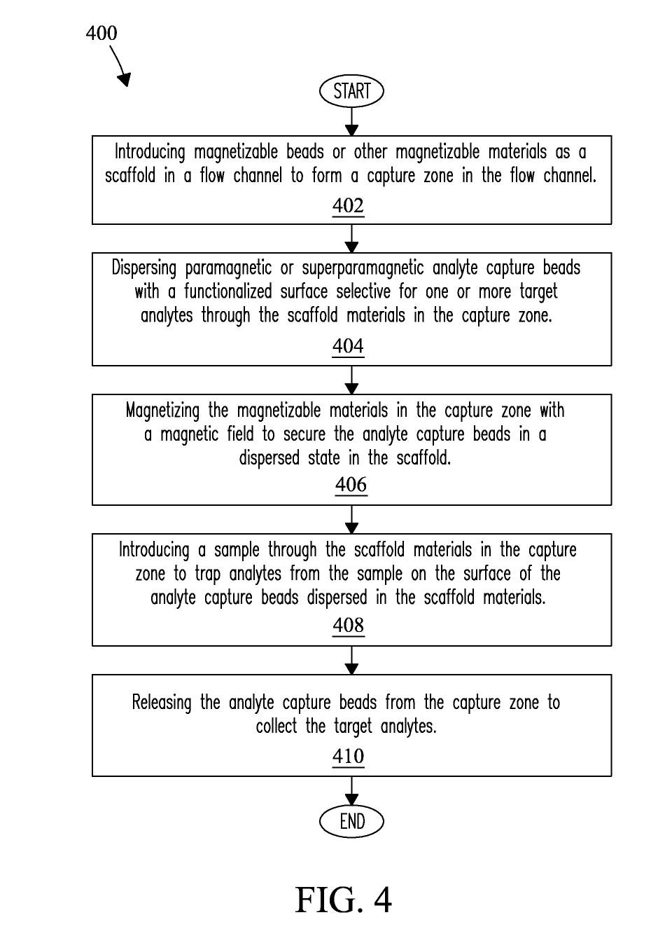

FIG. 4 presents an exemplary process 400 for selective capture of target analytes in a magnetizable bead trap and flow system 100 of the present invention. First (402), a quantity of magnetizable (scaffolding) beads 18 may be introduced into bead trap 12 in capture zone 14 within the flow channel 10, optionally in combination with other magnetizable scaffolding materials described previously herein in reference to FIG. 1. Next (404), a volume of paramagnetic or superparamagnetic analyte capture beads 26 dispersed or suspended in a carrier fluid 22 may be introduced into trap 12 and dispersed or distributed uniformly throughout capture zone 14. For example, analyte capture beads 26 may be introduced into trap 12 uniformly dispersed in a carrier liquid 22 at volumes so as to be uniformly dispersed throughout the scaffold of magnetizable beads (scaffold) 18 or other magnetizable materials 20 within trap 12. However, volumes are not intended to be limited. All volumes as will be selected by those of ordinary skill in the art in view of the disclosure are within the scope of the invention. No limitations are intended. Flowing analyte capture beads 26 into capture zone 14 in a uniformly dispersed state can assist retention of the paramagnetic or superparamagnetic beads 26 in a uniformly dispersed state as they enter magnetizable materials 18 or 20 present within capture zone 14. Flow rates and direction of flow are not intended to be limited. In some embodiments, analyte capture beads 26 can be used that are configured for capture of a single target analyte. In various embodiments, mixtures of analyte capture beads 26 configured for capture of single, multiple, or various target analytes may be used. And, unique combinations of individual target analyte capture beads 26 (e.g., each in a separate container or vessel) may be employed. No limitations are intended.

Next (406), a magnetic field may be delivered in concert with a magnet 40 or magnetic assembly 40 across the capture zone 14 within the flow channel 10 to magnetize the magnetizable scaffolding beads (scaffold) 18 or other scaffolding materials 20 within trap 12. Magnetization of the magnetizable scaffolding beads 18 and/or other magnetizable scaffolding materials 20 in the capture zone 14 secures paramagnetic or superparamagnetic analyte capture beads 26 in a distributed and uniformly dispersed state within scaffold of scaffolding beads 18. In the distributed and uniformly dispersed state, paramagnetic or superparamagnetic analyte capture beads 26 can enhance mass transport, capture, and capture efficiency of target analytes in samples 24 when introduced through the magnetizable scaffolding beads 18 or other scaffolding materials 20 in capture zone 14.

Next (408), a sample 24 containing a potential target analyte or target analytes may be introduced into trap 12 and flowed through the magnetizable scaffolding beads 18 or other scaffolding materials 20 within capture zone 14. Sample flow rates and flow direction are not intended to be limited. For example, flows through capture zone 14 may be provided in any combination of forward and reverse directions, at selected rates, and times sufficient to maximize attachment and capture of target analytes to surfaces of paramagnetic or superparamagnetic analyte capture beads 26 distributed and uniformly dispersed within the magnetizable bead scaffold 18 and/or other magnetizable materials 20 within capture zone 14.

Then (410), analyte capture beads 26 may be released from the trap 12 in the capture zone 14 and collected. The collection may also result in collection of the target analyte or analytes.

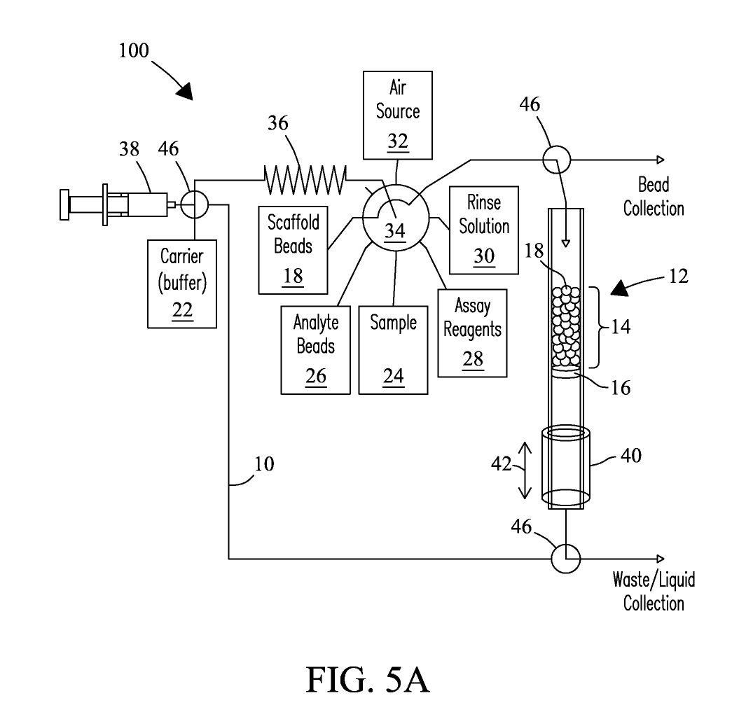

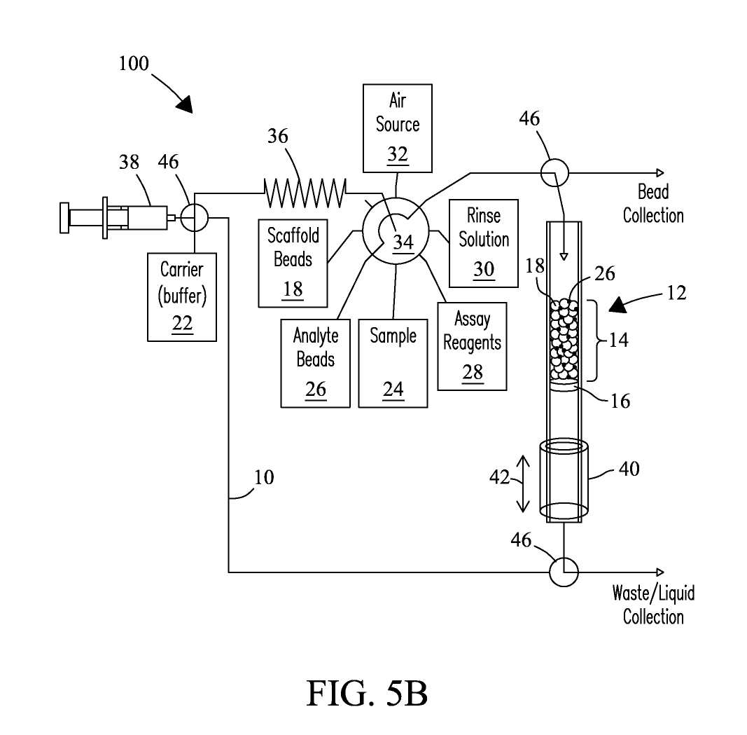

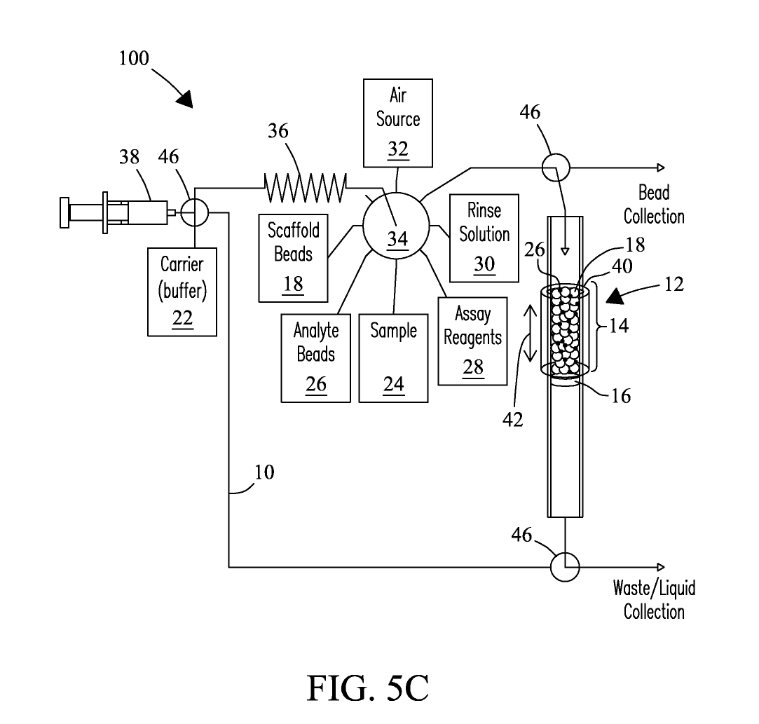

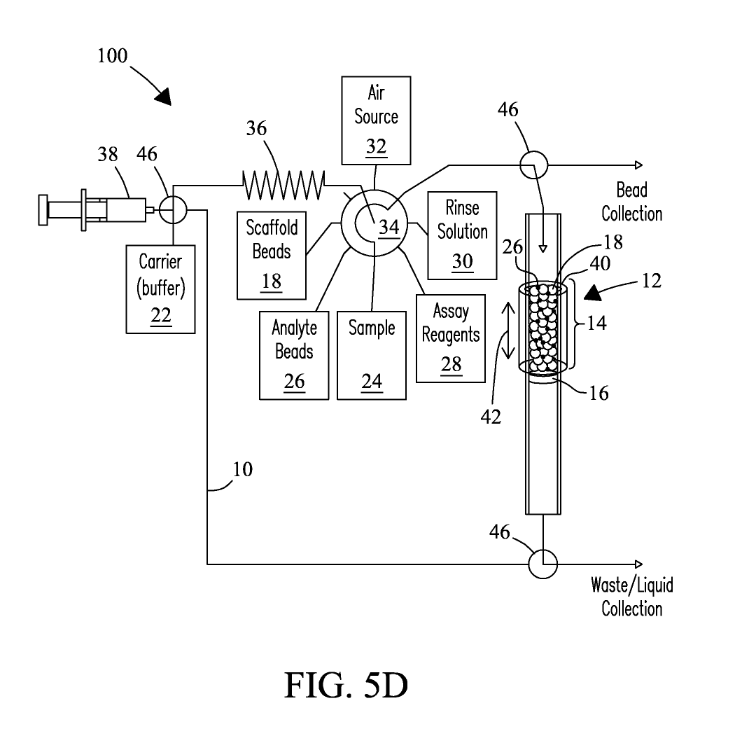

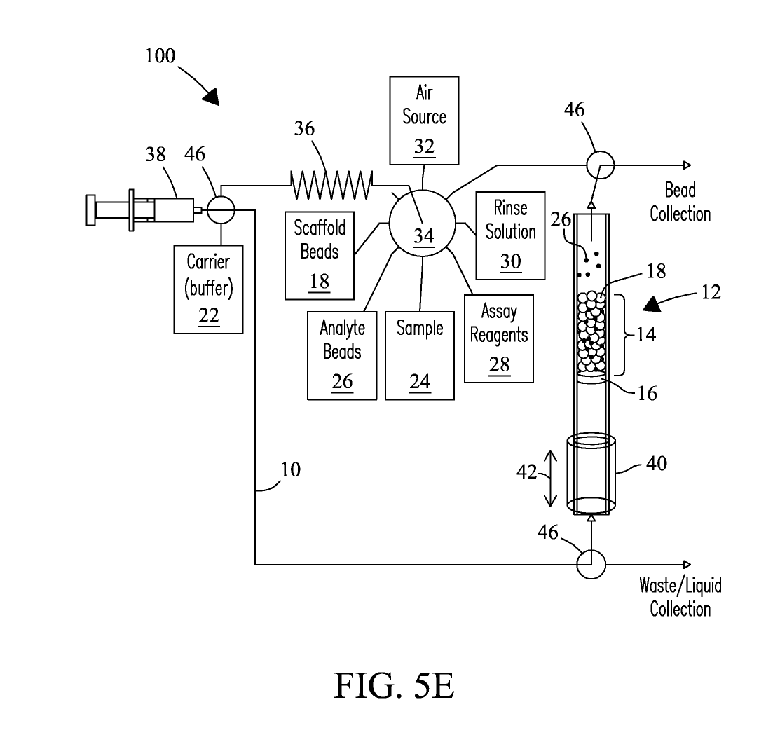

FIG. 5a illustrates the introduction of magnetizable (scaffold) beads 18 into bead trap 12 within capture zone 14. Alternatively, other magnetizable materials 20 may be pre-loaded in capture zone 14 and used as a scaffold 20 (see discussion in reference to FIG. 1b). No limitations are intended. In some embodiments, nickel beads or nickel coated beads 18 serve as the magnetizable scaffold for capturing analyte beads 26. In other embodiments, the magnetizable scaffold 18 or 20 may be composed of another metal-containing material that includes cobalt.

FIG. 5b illustrates the introduction of paramagnetic or superparamagnetic analyte capture beads 26 into bead trap 12. In the figure, a volume of paramagnetic or superparamagnetic analyte capture beads 26 dispersed in a carrier fluid 22 or suspension may be introduced into bead trap 12 and dispersed or distributed uniformly throughout the capture zone 14 within flow channel 10. Surface functionalized analyte beads 26 can provide capture of target analytes including, but not limited to, e.g., biological analytes, chemical analytes, radionuclide analytes, and other analytes. Analyte capture beads 26 may be paramagnetic or superparamagnetic beads or particles so as to be strongly attracted to magnetizable scaffold beads 18 and/or materials 20 in the presence of an applied magnetic field. Analyte capture beads 26 may be suspended in a selected buffer 22 or other carrier fluid 22, and aspirated, e.g., by a pump 38, and subsequently delivered into bead trap 12. In some embodiments, analyte capture beads 26 may be uniformly dispersed in a volume of carrier liquid 22 equal to the interstitial volume of the bead trap 12, to uniformly disperse analyte capture beads 26 through the magnetizable scaffold beads (scaffold) 18 or other magnetizable materials 20 within trap 12. Carrier fluid volumes are not limited. All volumes as will be selected by those of ordinary skill in the art in view of the disclosure are within the scope of the invention. No limitations are intended. As shown in the figure, prior to delivery, paramagnetic or superparamagnetic analyte capture beads 26 may be contained in a vessel (e.g., in a carrier liquid) to maintain the beads in a fully dispersed state or in a state that can be readily re-suspended to a fully dispersed state for immediate use in the fluidic system. Analyte capture beads 26 may be easily re-suspended by manually shaking the bead vessel, or by mixing the beads in the vessel, e.g., with an automated pump-based mixing device or protocol, or another mixing device known in the art. In some embodiments, a pump may function as a mixer and may repeatedly aspirate and dispense (re-introduce) equivalent volumes of the suspension liquid from the analyte capture bead vessel to achieve complete suspension of the analyte capture beads in the carrier liquid after several aspiration/dispensing cycles. Once suspended, paramagnetic or superparamagnetic analyte capture beads 26 may be aspirated into the fluidic system and delivered into capture zone 14 in concert with pump 38 or other pumping devices known in the art. In some embodiments, the selected mixer may repeatedly aspirate and dispense (reintroduce) equivalent volumes of the suspension liquid from the analyte capture bead vessel to achieve complete suspension of the analyte capture beads in the carrier liquid after several aspiration/dispensing cycles. Once suspended, paramagnetic or superparamagnetic analyte capture beads 26 may be aspirated into the fluidic system and delivered into capture zone 14 in concert with pump 38 or other pumping devices known in the art.

FIG. 5c illustrates positioning of a magnet 40 that locks analyte capture beads 26 in their uniformly dispersed state within trap 12. A magnetic field (not shown) may be applied across capture zone 14 within flow channel 10 in concert with a magnet 40 or magnetic assembly 40 that magnetizes the magnetizable scaffolding beads 18 or other scaffolding materials 20 within trap 12. As illustrated in the figure, magnet 40 may be positioned over bead trap 12 to rapidly (e.g., ˜200 milliseconds or less) magnetize the magnetizable scaffold beads 18. Magnetization of the magnetizable scaffolding beads 18 or other scaffolding materials 20 in the capture zone 14 can serve to secure paramagnetic or superparamagnetic analyte capture beads 26 in a distributed and uniformly dispersed state within the magnetizable scaffold bead 18 volume (i.e., both the cross section and length) within capture zone 14. In the uniformly dispersed state, analyte capture beads 26 enable maximum mass transport of target analytes in a sample 24 introduced through the scaffolding beads 18 volume or other scaffolding materials 20 in capture zone 14. Results show the present invention can provide: a two-fold to four-fold greater capture efficiency for capture of target analytes from samples 24 flowed through trap 12, and a five-fold to ten-fold faster capture of target analytes.

FIG. 5d illustrates capture of target analytes present in a sample 24. Here, a sample 24 may be flowed through the scaffold of magnetizable beads 18 or magnetizable materials 20 in trap 12 that contains analyte capture beads 26 in a uniformly dispersed state within the scaffold that is locked or fixed in concert with magnet 40. Sample flow rates are not limited. For example, flow rates are employed for a time sufficient to capture target analytes on the surface of paramagnetic or superparamagnetic analyte capture beads 26.

### Flow Control

In various embodiments, samples 24 can be flowed over paramagnetic or superparamagnetic analyte capture beads 26 uniformly distributed in the magnetizable bead scaffold 18 in various ways to maximize the quantity of target analytes captured from a sample 24, to improve efficiency of capture, and/or to minimize time required to capture analytes. In some embodiments, a sample 24 may be flowed multiple times through the magnetizable scaffold 18 or 20 containing analyte capture beads 26 in a repeated flow and stop mode to improve efficiency of analyte capture.

In some embodiments, a selected volume of sample may be flowed in a forward direction through scaffold 18 or 20 containing the analyte capture beads 26. Then the flow can be reversed and a volume of sample 24 less than the forward flow volume may be passed through the scaffold 18 or 20. The process may be repeated until the entire volume of sample 24 has been flowed over the paramagnetic or superparamagnetic analyte capture beads 26.

In some embodiments, sample 24 can be flowed in a forward direction. Then, when flow is reversed, the magnetic field may be removed to release paramagnetic or superparamagnetic analyte capture beads 26 into solution thereby improving analyte capture due to the solution phase kinetecs when the analyte capture beads 26 are in solution. Then, the magnetic field may be re-applied at an optimal time during forward flow to recapture the paramagnetic or superparamagnetic analyte capture beads 26 within the magnetizable bead scaffold 18 or magnetizable material scaffold 20 in capture zone 14. The volume of forward and reverse flow may be dependent on the geometry of capture zone 14. Timing of when to apply or re-apply the magnetic field may be dependent on the geometry of capture zone 14.

Paramagnetic and superparamagnetic analyte capture beads 26 uniformly distributed within magnetizable bead scaffold 18 or magnetizable material scaffold 20 in capture zone 14 may be washed, cleaned, and/or rinsed with rinse solution 30 and/or assay reagents 28 after perfusing sample 24 past paramagnetic and/or superparamagnetic analyte capture beads 26. Magnetizable scaffolds 18 or 20 provide an effective means for enhanced contact with and mass transport of assay reagents 28 to the paramagnetic or superparamagnetic analyte capture beads 26 and thus more rapid reaction rates and more efficient and complete washing, as no manual pipetting, centrifugation, or manual magnetic separation steps are required. Washing, rinsing, and reaction of paramagnetic or superparamagnetic analyte capture beads 26 with assay reagents 28 may employ a carrier 22 or a buffer 22 solution, but may also involve other reagents 28 and/or solutions as required by the selected assay(s). Washing may include one or more steps that enhance removal of excess assay reagents 28 or other potentially interfering or dissolved components, and/or otherwise unwanted particulate matter.

FIG. 5e illustrates one exemplary approach for releasing magnetizable scaffold beads 18 and/or paramagnetic or superparamagnetic analyte capture beads 26 from trap 12 to collect the analyte capture beads 26. In some embodiments, paramagnetic or superparamagnetic analyte capture beads 26 may be released from the scaffold 18 or 20 by removing the magnetic field and recapturing the analyte beads 26 upstream or downstream. Recapturing the analyte beads 26 may include reversing the flow of carrier fluid 22 or buffer fluid 22 through capture zone 14 in the absence of the magnetic field to release analyte beads 26 from their positions in the magnetizable scaffold 18 or 20, and then initiating a forward flow of carrier (buffer) fluid 22 or assay reagents 28 to again uniformly distribute and re-seat the paramagnetic or superparamagnetic analyte capture beads 26 within the interstitial spaces between the scaffold beads 18 or other magnetizable materials 20 in trap 12. The magnetic field may then be re-applied to lock paramagnetic or superparamagnetic analyte capture beads 26 in a uniformly dispersed state within the magnetizable scaffold 18 or 20 in trap 12.

In some applications, paramagnetic or superparamagnetic analyte capture beads 26 can be separated from scaffold beads 18 or other scaffold materials 20 by removing the magnetic field and flowing the analyte beads 26 through the pores of filter 16 or through the gap around rotating rod 50. Pore sizes in filter 16 or the gap widths around rod 50 may be selected to permit the flow of analyte beads 26 through the pores or the gap. Pore sizes and gap widths may be equal to or greater (e.g., 2-10 times greater) than the diameter of the analyte capture beads 26.

In some applications, paramagnetic or superparamagnetic analyte capture beads 26 can be separated from the magnetizable scaffold 18 or 20 in the capture zone 14 without removing the magnetic field. Due to their smaller size, or by selecting different materials of composition, analyte capture beads 26 can have a lower magnetic susceptibility than magnetizable scaffold beads 18 or other magnetizable scaffold materials 20 in the capture zone 14. Thus, by flowing analyte capture beads 26 (e.g., with their captured target analytes) at a sufficiently high flow rate, analyte capture beads 26 may be released from the magnetizable scaffold 18 or 20 and collected upstream or downstream from trap 12 even when the magnetic field in capture zone 14 remains.

In some applications, paramagnetic or superparamagnetic analyte capture beads 26 may be released from scaffold 18 or 20 in trap 12 by increasing the rate of flow or volume of carrier fluid 22 through trap 12 downstream (e.g., through the rotatable rod) or upstream while the magnet 40 (and magnetic field) and scaffold 18 or 20 remain in place in trap 12. Paramagnetic or superparamagnetic analyte capture beads 26 when released may then be collected upstream or downstream from trap 12.

In some applications, both magnetizable scaffold beads 18 or magnetizable scaffold materials 20 and analyte capture beads 26 may be released together from trap 12 by removing the magnetic field and flowing the scaffold beads 18 and capture beads 26 upstream or downstream from trap 12. After collection, scaffold beads 18, other magnetizable scaffold materials 20, and analyte capture beads 26 may be separated external to trap 12, e.g., using physical separation, magnetic separation, or buoyancy differences between the analyte capture beads 26 and the scaffold beads 18 or other magnetizable scaffold materials 20. For example, when the magnetic field is removed from trap 12, buoyancy of scaffold beads 18 can assist their collection upstream from trap 12, while paramagnetic or superparamagnetic analyte capture beads 26 may proceed downstream and be captured, e.g., in concert with a magnet positioned downstream, or vice versa.

In some applications, paramagnetic or superparamagnetic analyte capture beads 26 may be released from the scaffold (beads) 18 by removing the magnetic field and recapturing the analyte capture beads 26 upstream or downstream. Recapturing the analyte beads 26 may include reversing the flow of carrier (or buffer) fluid 22 through capture zone 14 in flow channel 10 in the absence of the magnetic field to release analyte capture beads 26 from their position within scaffold 18, and then initiating a forward flow of carrier (or buffer) fluid 22 to again uniformly distribute and re-seat the paramagnetic or superparamagnetic analyte capture beads 26 within the interstitial spaces between the magnetizable scaffold beads 18 in trap 12.

In some applications, paramagnetic or superparamagnetic analyte capture beads 26 may be released from trap 12 in capture zone 14 by removing the magnetic field, e.g., by returning the ring magnet 40 to an original position below capture zone 14, or by de-energizing the electromagnet 40, or by altering the flow state within capture zone 14 and collecting the paramagnetic or superparamagnetic analyte beads 26 containing the target analytes.

A cleavable ligand on the surface of the paramagnetic or superparamagnetic analyte capture beads 26 can also be employed to enable release of captured target analytes from the analyte capture beads 26. The cleavable ligand can be cleaved, e.g., in concert with ultraviolet light or a chemical or a cleaving enzyme as will be understood by those of ordinary skill in the biochemical arts. In some embodiments, captured target analytes such as bacterial cells can be lysed while still attached to the analyte capture beads 26, thus allowing release and separation of internal cellular components such as DNA and RNA from the analyte capture beads 26 for subsequent collection, purification, analysis, or detection. In some embodiments, analyte capture beads 26 with bacteria attached to the analyte capture beads 26 can be collected and cultured without separation of the bacteria from the analyte capture beads 26, e.g., on agar plates or other suitable growth media. All approaches as will be employed by those of ordinary skill in the art in view of the disclosure are within the scope of the present invention. No limitations are intended.

### Target Analytes

Target analytes include, but are not limited to, chemical analytes, biological analytes, radiological analytes, or combinations of these various analytes detailed herein. Chemical analytes can include, but are not limited to, e.g., metals; heavy metals including, e.g., cobalt (Co), copper (Cu), silver (Ag), arsenic (As), cadmium (Cd), chromium (Cr), mercury (Hg), lead (Pb), selenium (Se), thallium (TI), germanium (Ge), yttrium (Y), indium (In), iron (Fe), iridium (Ir); and other heavy metals; explosives; explosive precursors; chemical nerve agents; toxic industrial chemicals (TICs); poisonous compounds (e.g., pesticides, herbicides, rodenticides, and like compounds); flammable compounds; fuel components; oxidizing chemicals; reducing chemicals; corrosive (e.g., caustic and acidic) agents; and combinations of these various chemical analytes. Radionuclides may include, but are not limited to, e.g., actinides, lanthanides, and/or other radionuclides including, e.g., cesium (Cs), uranium (U), Americium (Am), cobalt (Co), technetium (Tc), tritium, thorium (Th), plutonium (Pu), strontium (Sr), radium (Ra), iodine (I), neptunium (Np), neodymium (Nd), samarium (Sm), dysprosium (Dy). Biological analytes can include, but are not limited to, e.g., cells, proteins, protein toxins (e.g., ricin, botulinum neurotoxin, Staphylococcus enterotoxin-B), lipids, carbohydrates, quorum sensing molecules, growth inducing molecules, metabolites, auto-inducers, nucleic acids including DNA and RNA; bacteria, viruses, oocytes, spores (e.g., Bacillus anthracis), celled organisms, other biological materials, and combinations of these various materials. In some embodiments, the biological analyte may be a virus such as a retrovirus. In some embodiments, the biological analyte may be a bacterium such as, e.g., Listeria; E. coli; Salmonella, including combinations of these bacterial types at both the genus and species level. No limitations are intended.

### Chemically Selective Functional Groups

In various applications, surfaces of paramagnetic and superparamagnetic analyte capture beads may be functionalized with functional groups (ligands) that are chemically selective for target analytes of interest. Target analytes include, but are not limited to, e.g., chemicals, metals, and radionuclides. Functional groups (ligands) include, but are not limited to, e.g., thiols, lauric acids, ethylenediamine tetraacetic acid (EDTA), L-glutathiones, mercaptobutyric acids, meso-2,3-dimercaptosuccinic acids, ferric-potassium hexacyanoferrates, manganese dioxide, and combinations of these various functional groups. In some embodiments, functional groups selected for capture of biological analytes may include, but are not limited to, e.g., antibodies, oligonucleotides, DNA, RNA, lectins, carbohydrates, hydroxyapatite, silica, streptavidin, biotin, including combinations of these various functional groups.

In some embodiments, functional groups selected for capture of heavy metals and radionuclides may include, but are not limited to, e.g., monodentate ligands, bidentate ligands, polydentate ligands, chelate rings (e.g., 5-member), inorganic chelate ligands, hexadentate chelate ligands, undecanoic acids, thiols, ethylene diamine tetraacetic acid (EDTA); MBA; desferrioxamine (DFOA); dimercaprol; dimercapto succinic acid (DMSA); dimercaptopropane sulfonate (DMPS); MnO₂; analogs of these various chelators and ligands, including combinations of these various functional groups. All functional groups as will be employed by those of ordinary skill in the chemical or separation arts are within the scope of the present invention.

### Analyte Selectivity

Analyte selectivity is a function of the surface chemistry of the paramagnetic and superparamagnetic analyte capture beads used to capture the target analytes in the capture zone. The present invention provides significant improvements over traditional analyte capture approaches including, e.g., mechanical mixing where analyte capture beads can clump on the side-wall of a flow channel, or where analyte capture beads in iron wool do not distribute uniformly, or where iron wool retains residual magnetism when a magnetic field is removed. The present invention provides up to 500% faster capture of target analytes such as cytokines on immunomagnetic beads in the capture zone. In addition, 200% to 300% more complete capture of target analytes has been demonstrated when using immunomagnetic beads that are uniformly distributed in a magnetizable scaffold in the capture zone as compared to using no magnetizable scaffold or capture zone. Advantages of the present invention result from: 1) improved mass-transport, because target analytes in a sample may be actively flowed over the analyte capture beads as compared with passive diffusion in conventional mechanical mixing; 2) effective concentration of all analyte capture beads in a uniformly dispersed state in a small volume (i.e., within a capture zone) such that at any one point in time, only a small sub-portion of the overall sample volume is in contact with all of the analyte capture beads. In contrast, in traditional mechanical mixing and assays, analyte capture beads are “diluted” in the presence of the entire sample volume; and 3) flowing the sample over uniformly dispersed analyte capture beads in a scaffold assists in “forcing” contact between target analytes and the paramagnetic and superparamagnetic analyte capture beads.

### Sample Volumes

Typical sample volumes in concert with the present invention range from about 1 microliter (μL) to greater than about 1000 liters (L). In some embodiments, sample volumes are less than about 10 microliters (μL). In some embodiments, sample volumes are greater than about 10 microliters (μL). In some embodiments, sample volumes are greater than 1 milliliter (mL). In some embodiments, sample volumes are greater than or equal to about 1000 mL. In some embodiments, sample volumes are between about 1 mL and 1000 mL. In some embodiments, sample volumes can be 1 L to greater than 1000 L. However, volumes are not intended to be limited.

### Analytical Approaches

Analytical methods suitable for detection and analysis of target analytes captured on functionalized paramagnetic or superparamagnetic analyte capture beads in the scaffold bead trap are not limited. Exemplary methods include, e.g., Flow Cytometry, On-column Fluorescence, sandwich immunoassays, enzyme linked immunoassays (ELISA), polymerase chain reaction (PCR), and sequencing. However, all analytical methods as will be employed by those of ordinary skill in the art for detection of target analytes in view of the disclosure are within the scope of the present invention. No limitations are intended.

Detectors suitable for detection of target analytes captured on functionalized paramagnetic or superparamagnetic analyte capture beads in the scaffold bead trap may include, but are not limited to, e.g., biochip detectors; flow cytometry detectors; polymerase chain reaction (PCR) detectors; DNA sequencing instruments and detectors; mass-selective detectors such as, e.g., Selected Ion Flow Tube (SIFT) mass spectrometers (SIFT-MS), Proton Transfer Reaction Mass Spectrometers (PTR-MS), and Atmospheric Pressure Chemical Ionization Mass Spectrometers (APCI-MS); charge-coupled device (CCD) detectors; fluorescence detectors; ultraviolet detectors; visible detectors; Raman spectrometry instruments and detectors; Fourier Transform Infrared (FTIR) spectrometry instruments and detectors; electrochemiluminescence (ECL) instruments and detectors; inductively coupled plasma spectrometry (ICP) instruments and detectors; inductively coupled plasma mass spectrometry (ICP-MS) instruments and detectors; atomic absorption (AA) instruments and detectors; X-ray fluorescence (XRF) instruments and detectors; optical emission spectrometry (OES) instruments and detectors; direct current (DC) spark instruments and detectors; electrochemical instruments and detectors; colorimetric instruments and detectors; voltammetry instruments and detectors; amperometry instruments and detectors; surface acoustic wave instruments and detectors; imaging variants of these various instrument detector systems and detectors; including components and combinations of these various detectors and instrument systems. No limitations are intended.

### Applications

The present invention finds application in food safety and other material safety applications, microbiological testing applications, homeland security and defense applications, military and force protection applications, field screening applications, bio-surveillance and bio-monitoring applications, bio-threat detection applications, clinical diagnostic applications, sample preparation applications, and other like applications. No limitations are intended.

### Example

### Immunomagnetic Capture of E-coli

The system of FIG. 1 was used. Enhanced capture of E-coli 0157:H7 was demonstrated in 50 mL sample volumes. Superparamagnetic anti-E-coli 0157:H7 immunomagnetic “Dynabeads” were used (Invitrogen, Carlsbad, Calif.). E-coli concentration was approximated by measuring the optical density at 600 nm, but colony forming units (CFUs) per mL of all samples were determined each day by plate count analysis after culturing overnight at 37° C. on agar plates. Capture efficiency was assessed by culturing supernatant from a sample containing superparamagnetic analyte capture beads before and after processing. Positive control samples included samples containing E-coli with no added beads and were used to establish the actual number of CFU per mL of solution. E-coli was cultured by plating eight 20 microliter spots for each sample. Negative control samples (no E-coli added to capture beads) showed no contamination (no colony growth). Manual bench top assays were performed in a 50 mL tubes by invert mixing for 1 hour at room temperature. Samples for automated fluidics assays were prepared in the same manner as manual benchtop assays, then circulated through a microparticle flow trap containing a magnetizable scaffold material composed of a magnetizable nickel foam (Porvair, Norfolk, UK) having a non-limiting porosity of 80 pores per inch. Flow through the trap was accomplished using a peristaltic pump using an exemplary flow rate of 32 mL/min. Volume of the sampling line tubing (inside diameter of 2.8 mm) from inlet to outlet was 10 mL. Magnetizable nickel foam piece forming the capture zone with the trap was 20-mm in length and 4.75-mm in diameter. Six NdFeB ring magnets (Part #0056; Forcefield, Fort Collins, Colo., USA) surrounded the trap. Each ring magnet was 6.35 mm in diameter and 25.4 mm in length. Magnets surrounding the magnetizable nickel foam provided a magnetic flux within the flow trap that provided a high magnetic field gradient across the width and length of the capture zone, allowing uniform distribution of superparamagnetic analyte capture beads uniformly dispersed through the volume of the flow trap. After 1 hour of recirculating the 50-mL sample, a portion of the liquid was removed for culturing to assess whole-cell capture efficiency of the antibody-coupled analyte capture beads. After processing, magnets were removed and analyte capture beads were flushed to a capture vessel. Nickel foam was reused for subsequent samples after sterilization using 0.5% bleach, followed by rinsing with deionized water. Blanks (containing no bacteria) were processed through the automated system and culturing confirmed that there was no carryover of bacteria in the fluidics system after processing samples. Results showed that the fluidics system configured with a magnetizable nickel foam scaffold material and superparamagnetic analyte capture beads resulted in a capture of 43% of E. coli cells. By comparison, a conventional fluidic system employing no nickel foam resulted in a capture of 19%. Also, by comparison, a conventional manual bench top analysis using invert mixing resulted in a capture of 12%. Other aspects of the approach are detailed by Ozanich et al. in J. Lab. Automation, 12(5), 303-310 (2007), which reference is incorporated herein in its entirety.

While exemplary embodiments of the present invention have been shown and described, the invention is not intended to be limited thereto. For example, from the description, it will be apparent to those skilled in the art that many changes and modifications, alterations, and substitutions may also be made without departing from the true scope of the invention in its broader aspects. The appended claims are therefore intended to cover all such changes and modifications as fall within the scope of the present invention.

What is claimed is:

1. A method for capture of one or more target analytes, the method comprising the steps of:
introducing a quantity of magnetizable beads of a selected size or another magnetizable material at a selected location within a flow channel that defines a capture zone to form a magnetizable scaffold therein;
dispersing a quantity of paramagnetic or superparamagnetic analyte capture beads having a functionalized surface selective for capture of one or more target analytes thereon through the magnetizable scaffold to uniformly disperse same therein;
magnetizing the magnetizable scaffold to secure the paramagnetic or superparamagnetic analyte capture beads in the uniformly dispersed state in the magnetizable scaffold; and
introducing a sample containing one or more target analytes through the magnetizable scaffold in the capture zone to trap the target analytes on the surface of the paramagnetic or superparamagnetic analyte capture beads uniformly dispersed therein.

2. The method of claim 1, wherein introducing the other magnetizable material includes pre-loading a magnetizable material selected from the group consisting of: metal foams, metal wools, metal mesh, metal wires, and combinations thereof into the trap to form a magnetizable scaffold therein having a selected porosity prior to uniformly dispersing the paramagnetic or superparamagnetic analyte capture beads through the magnetizable scaffold in the capture zone.

3. The method of claim 1, wherein introducing the magnetizable beads includes retaining the magnetizable beads in the capture zone with a rotatable rod disposed at one end of the capture zone, the rod including a beveled face that in first position retains the scaffold beads in the capture zone and in a second position releases the scaffold beads from the capture zone.

4. The method of claim 1, wherein the dispersing includes uniformly dispersing the paramagnetic or superparamagnetic analyte capture beads into interstitial spaces disposed between the magnetizable beads of the scaffold

5. The method of claim 1, wherein the dispersing includes uniformly dispersing the paramagnetic or superparamagnetic analyte capture beads across the cross-section and length of the scaffold in the capture zone.

6. The method of claim 1, wherein the dispersing includes uniformly dispersing the paramagnetic or superparamagnetic analyte capture beads in a volume of fluid approximately equal to the volume of the magnetizable beads that form the scaffold in the capture zone.

7. The method of claim 1, wherein the dispersing includes uniformly dispersing the analyte capture beads in a volume of fluid less than or equal to the volume of magnetizable beads within the capture zone.

8. The method of claim 1, wherein the dispersing includes uniformly dispersing the paramagnetic or superparamagnetic analyte capture beads within the scaffold in the capture zone prior to flowing a sample containing the one or more analytes through the scaffold in the capture zone.

9. The method of claim 1, wherein the dispersing includes uniformly dispersing the paramagnetic or superparamagnetic analyte capture beads in a uniformly dispersed state and the sample containing the one or more target analytes through the scaffold in the capture zone simultaneously.

10. The method of claim 1, wherein magnetizing the magnetizable scaffold beads is performed with a magnet assembly comprising at least one magnet.

11. The method of claim 10, wherein the magnet assembly includes a first position that secures the paramagnetic or superparamagnetic analyte capture beads in a uniformly dispersed state in the scaffold and a second position that releases the paramagnetic or superparamagnetic analyte capture beads from the scaffold within the capture zone in a selected flow direction.

12. The method of claim 1, wherein flowing the sample through the capture zone includes a flow rate that provides a residence time sufficient for capture of the one or more target analytes on the surface of the paramagnetic or superparamagnetic analyte capture beads uniformly dispersed in the scaffold in the capture zone.

13. The method of claim 1, further including collecting the paramagnetic or superparamagnetic analyte capture beads containing the one or more target analytes thereon from the capture zone for analysis of the target analytes.

14. The method of claim 13, wherein collecting the paramagnetic or superparamagnetic analyte capture beads from the capture zone includes flowing a fluid through the capture zone in a forward flow direction, a reverse flow direction, or a combination of a forward flow direction and a reverse flow direction.

15. The method of claim 13, wherein collecting the paramagnetic or superparamagnetic analyte capture beads from the capture zone includes maintaining the magnetic field to secure the magnetizable beads in the scaffold and flowing a fluid through the scaffold to release the paramagnetic or superparamagnetic analyte capture beads from the scaffold.

16. A trap and flow system for selective capture of a target analyte, comprising:
a quantity of paramagnetic or superparamagnetic analyte capture beads of a selected size uniformly dispersed within a magnetizable scaffold comprising of larger magnetizable beads or another magnetizable material at a selected location in a flow channel that defines a capture zone; and
a magnet disposed to magnetize and secure the analyte capture beads within the magnetizable scaffold in the capture zone;
whereby the analyte capture beads are configured to capture a selected analyte when a sample containing a selected analyte is introduced in through the magnetizable scaffold in the capture zone.

17. The system of claim 16, wherein the other magnetizable material is selected from the group consisting of: metal wools, metal meshes, metal wires, metal filaments, and combinations thereof configured to maintain pores of a selected size in the magnetizable scaffold that serves to uniformly disperse the paramagnetic or superparamagnetic analyte capture beads within the scaffold in the capture zone.

18. The system of claim 16, wherein the magnet is disposed on a translational stage.

19. The system of claim 18, wherein the translational stage is configured to position the magnet in a first position proximate the capture zone that magnetizes the scaffold and secures the paramagnetic or superparamagnetic analyte capture beads in a uniformly dispersed state within the scaffold, and a second position a selected distance removed from the capture zone that releases the analyte capture beads from the scaffold in the capture zone for collection of the one or more target analytes captured thereon.

20. The system of claim 16, wherein the magnet applies a magnetic field in a direction orthogonal to the flow of the carrier fluid in the capture zone.
